(19) United States
(12) Patent Application Publication
(10) Pub. No.: US 2011/0052688 A1
(43) Pub. Date: Mar. 3, 2011

(54) SOLID DISPERSION COMPOSITION

(76) Inventors: San-Laung Chow, San Jose, CA (US); David Wong, Milpitas, CA (US); Edward Lin, Fremont, CA (US)

(21) Appl. No.: 12/907,388

(22) Filed: Oct. 19, 2010

Related U.S. Application Data
(63) Continuation-in-part of application No. 11/943,386, filed on Nov. 20, 2007.
(60) Provisional application No. 60/866,812, filed on Nov. 21, 2006.

(51) Int. Cl. A61K 9/24; A61K 31/405

(52) U.S. Cl. 424/472; 514/419

(57) ABSTRACT

A solid dispersion extended release tablet composition is provided. The composition comprises fluvastatin sodium and a polymer, wherein molecules of the fluvastatin sodium are separate from one another and dispersed in the polymer, and wherein the composition displays two distinct peaks at about 3.5 and 20.4.degrees 2θ of X-ray diffraction.

### CROSS-REFERENCE TO RELATED APPLICATIONS

This application is a continuation-in-part of co-pending U.S. patent application Ser. No. 11/943,386, filed Nov. 20, 2007, which claims benefit of U.S. provisional patent application Ser. No. 60/866,812, filed Nov. 21, 2006, all of which are herein incorporated by reference in their entirety.

### BACKGROUND OF THE INVENTION

1. Field of the Invention

Embodiments of the invention relate to a solid dispersion composition suitable as a therapeutic agent and a pharmaceutical drug in a pharmaceutical composition that allows a zero-order drug release over a prolonged period of time.

2. Background Art

A solid dispersion is generally considered as a dispersion of one or more active ingredients in a carrier at a solid state. Generally, solid dispersion using tedious techniques such as water-in-oil emulsion is used to improve dissolvability in water of a water-insoluble drug or a poorly water-soluble drug in a pharmaceutical composition, to mask the taste of a drug substance, and/or to prepare rapid disintegration of oral tablets or sustained-release microspheres.

Fluvastatin sodium is a water-soluble cholesterol lowering agent which acts to inhibit 3-hydroxy-3-methylgutaryl-coenzyme A (MHMG-CoA) reductase. Fluvastatin sodium is a monosodium salt form of [R*,S*-(E)]=(±)-7-[3-(4-fluorophenyl)-1-(1-methylethyl)-1Hindol-2-yl]-3,5-dihydroxy-6-heptenoic acid. In patients with hypercholesterolemia and mixed dyslipidemia, treatments with fluvastatin sodium reduce the levels of total cholesterols, LDL-cholesterol, apoliporotein B, and triglycerides and increase the levels of HDL-cholesterol. Fluvastatin sodium is found to exist as different crystalline forms under various conditions and with different stabilities. Attempts have been made to incorporate different crystalline forms of fluvastatin sodium into sustained-release dosage forms.

However, most sustained release fluvastatin tablets were found to be unstable when exposed to light and undergo photo-degradation as observed by apparent change of colors after prolonged storage. Various ways to improve color stability of these crystalline forms of fluvastatin sodium and stabilize fluvastatin sustained release tablets were tried, including reduction of ambient moisture levels, reduction of mean granule particle size, and use of excessive amount of colorants.

For example, crystalline form of fluvastatin and hydroxypropyl methyl cellulose have been mixed to directly prepare fluvastatin into granules and then into sustained release tablets, using up to 12 percent of hydroxypropyl functional groups and an average molecular weight of about 20,000 to about 170,000 as the hydroxypropyl methyl cellulose in the sustained release tablets. Non-ionic hydrophilic polymers of hydroxypropyl cellulose or polyethylene oxide have also been used to mix in the granules of fluvastatin and hydroxypropyl methyl cellulose to prepare its sustained-release dosage form. As another example, crystalline form of fluvastatin was also found to mix with hydroxypropyl methyl cellulose at a molecular weight between about 20,000 and 30,000 and a nonionic hydrophilic polymer of hydroxyethyl cellulose or polyethylene oxide in order to improve its color stability.

Therefore, there exists a need for pharmaceutical compositions of a color-stable dosage form and a method for preparing such compositions.

### SUMMARY OF THE INVENTION

Embodiments of the invention generally provide pharmaceutical drug compositions, methods of preparing oral drug compositions, such as controlled release dosage compositions for one or more active ingredients, such as color-instable active ingredients. In one embodiment, a pharmaceutical composition having a mixture of one or more water soluble active ingredients and one or more pharmaceutical acceptable polymers dissolved by a solvent and prepared into a dispersion solution is provided. The pharmaceutical composition can be prepared into solid dosage forms by mixing the dispersion solution with a pharmaceutical acceptable controlled released polymer, a binder, and/or a lubricant via granulation.

In another embodiment, the mixture of one or more water soluble active ingredients and one or more pharmaceutical acceptable polymers in the pharmaceutical composition are melted at high temperature and blended before forming into solid dosage forms. In still another embodiment, one or more active ingredients are prepared into a solid dispersion composition. In still another embodiment, the pharmaceutical composition further includes a surfactant to facilitate dispersing of the water soluble active ingredients into the one or more pharmaceutical acceptable polymers such that the resulting dispersion solution can be melted at high temperature or dissolved by a solvent.

In still another embodiment, one or more active ingredients prepared into a solid dispersion composition are amorphous without any observed crystalline structures. Further, the pharmaceutical composition according to one or more embodiments of the invention is capable of providing a constant release rate, such as a substantially zero-order release rate, for the one or more amorphous active ingredients. In one example, a pharmaceutical composition includes a therapeutically active drug and a polymer material in a solid dispersion to achieve desired in vivo and in vitro performance, e.g., a constant in vitro drug dissolution profile. In addition, an effective amount of a non-toxic, pharmaceutically acceptable controlled release agent or polymer compound can be included to assist and modify the release rate of the therapeutically active drug. One example of a therapeutically active drug is fluvastatin and/or its salts thereof, such as fluvastatin sodium.

In yet another embodiment, a solid dispersion extended release tablet composition is provided. The composition comprises fluvastatin sodium and a polymer, wherein molecules of the fluvastatin sodium are separate from one another and dispersed in the polymer, and wherein the composition displays two distinct peaks at about 3.5 and 20.4.degrees 2θ of X-ray diffraction.

In yet another embodiment, a solid dispersion extended release tablet composition is provided. The composition comprises fluvastatin sodium and a polymer in a solidification of an at least partially liquid dispersion solution of the fluvastatin sodium and the polymer together, wherein at least some of both the fluvastatin sodium and the polymer are liquid in the at least partially liquid dispersion solution, wherein at least a portion of the fluvastatin sodium is dispersed in the polymer, wherein the fluvastatin sodium has an amorphous form in the solidification and wherein molecules of the fluvastatin sodium are separate from one another and dispersed in the polymer, wherein the composition enables a constant release rate of fluvastatin sodium for a period of about 12 hours.

In yet another embodiment, a solid dispersion extended release tablet composition, comprising fluvastatin sodium and a polymer, wherein molecules of the fluvastatin sodium are separate from one another and dispersed in the polymer, and wherein the corresponding Cₘₐₓ ratio of the composition with respect to LESCOL® XL is about 0.8-1.2.

In yet another embodiment, an extended release tablet composition, comprising (a) a granule comprising a dispersion and a substrate, (i) the dispersion comprising fluvastatin sodium and Hypromellose 2208, wherein molecules of the fluvastatin sodium are separate from one another and dispersed in the Hypromellose 2208 and wherein the viscosity of 2% solution of Hypromellose 2208 in water is 2,663-4,970 mPa·pmd s, and (ii) the substrate comprising cellulose gum and microcrystalline cellulose, wherein the dispersion and substrate are combined to form granules, and (b) extra-granular materials comprising glycerol monostearate, cellulose gum and polyethylene, wherein the extended release tablet composition displays two distinct peaks at about 3.5 and 20.4.degrees 2θ of X-ray diffraction and wherein the composition enables fluvastatin to be constantly released over a time period of about 12 hours is provided.

### BRIEF DESCRIPTION OF THE DRAWINGS

So that the manner in which the above recited features of the invention can be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIG. 1 illustrates, in accordance with one or more embodiments of the invention, drug release profiles of three exemplary pharmaceutical compositions.

FIG. 2 illustrates X-ray powder diffraction results of a pharmaceutical composition, in accordance with one or more embodiments of the invention, compared to a placebo.

FIG. 3 illustrates, in accordance with one or more embodiments of the invention, drug release of one exemplary pharmaceutical composition in comparison with a known pharmaceutical composition.

### DETAILED DESCRIPTION

Embodiments of the invention generally provide pharmaceutical drug compositions having one or more active ingredients dispersed in one or more pharmaceutical acceptable polymers or waxes and prepared into solid dosage forms. The one or more active ingredients can be dispersed into the one or more pharmaceutical acceptable polymers through various processes. For example, a solvent-based process, a fusion-melt process, a hybrid fusion-solvent process or other dispersion processes can be used to prepare one or more pharmaceutical active drug substances into solid dispersion. Both melting and solvent based techniques define approaches to dissolve one or both of the active ingredient and the polymer.

In one aspect, the solvent-based process uses a solvent, such as water, non-organic solvents, and organic solvents, to dissolve and intimately disperse or dissolve the drug and the one or more pharmaceutical acceptable polymers. The solvent is later removed by evaporation or other ways while the drug/polymer solid dispersion is collected into a solid dosage form. The use of organic solvents may generate hazardous and toxic wastes to the environment. If possible, water is used for water soluble drugs to prepare a dispersion. Other suitable solvents may be, for example, alcohols and acetone for the use of water-insoluble polymers. In addition, fluvastatin sodium was found to be soluble in water, alcohols and acetone, and thus can be dissolved, for example, in any of these solvents to prepare a dispersion.

The resulting dispersion for preparing the solid dosage forms can be mixed with additional polymers, controlled release agents, binders, lubricant, and/or fillers. For example, the resulting dispersion can be blended with a mixture of polymers, controlled release agents, binders, lubricant, and/or fillers, through granulation before compressing into tablets or other solid dosage forms.

In another aspect, the fusion-melt process involves melting the drug and the one or more pharmaceutical acceptable polymers together at temperatures at or above the melting point of either the one or more pharmaceutical acceptable polymers and/or the drug. In the fusion-melt process, the drug and one or more pharmaceutical acceptable polymers can first be blended and melted in a suitable mixer. The molten mixture is then cooled rapidly to provide a congealed mass. Alternatively, the one or more pharmaceutical acceptable polymers can be melted into a molten state before mixing with the drug into a homogeneous state. The melted mixture of the drug and the one or more pharmaceutical acceptable polymers may be congealed by lowering the temperatures and then prepared into pharmaceutical dosage forms, such as a solid dosage form, e.g., powder and tablets. For example, the cooled mixture can be subsequently milled to produce a powder form. Alternatively, the cooled mixture can be milled and blended with additional fillers, lubricant, and/or binders and compressed into tablets.

In still another aspect, the hybrid fusion-solvent process can be used. For example, if there is thermal instability and immiscibility between the drug and the one or more pharmaceutical acceptable polymers, the drug can initially be dissolved in a small quantity of a solvent and added to a molten pharmaceutical acceptable polymer. The solvent is then evaporated to generate a product that is subsequently milled to produce a solid dosage form, such as a powder form, or compressed into tablets.

Pharmaceutical compositions containing fluvastatin sodium and a variety of polymer components are investigated for desired tablet appearance and drug release after a one-month stability test. It is found that when fluvastatin sodium and a polymer are prepared into a dispersion composition in solid form, the tablet appearance of the solid dispersion can be remarkably enhanced. In addition, a desired constant controlled release profile or a sustained drug release profile can be achieved. Optionally, a surfactant can be used to prepare fluvastatin into dispersion. Surprisingly, it was also found that a solid dispersion composition of fluvastatin exhibits uniform color and exists in its amorphous form, and color stability is maintained even after a stability test for one month. Solid dosage forms of fluvastatin made out of direct compression appears to include noticeable yellow spots. As a comparison, fluvastatin prepare by dispersion before made into solid tablet forms by granulation exhibit no noticeable color spots after one-month stability test under accelerated conditions, even though their colors may be slightly darker.

In one embodiment, a solid dispersion composition comprising fluvastatin sodium and a polymer is provided. Optionally a surfactant is added into the solid dispersion composition. In another embodiment, a sustained release pharmaceutical composition comprising a solid dispersion of fluvastatin and/or its salts thereof, a pharmacologically acceptable polymer, and optionally a surfactant is provided. The solid dispersion composition may include fluvastatin in an amount of about 0.1 wt % to about 50 wt %, such as about 5 wt % to about 45 wt % of the total pharmaceutical composition.

In another embodiment, an extended-release tablet composition comprising: (a) about 84.5 mg of fluvastatin sodium, (b) hypromellose in a range of 2-35 percent by weight, (c) cellulose gum in range of 20-50 percent by weight, (d) microcrystalline cellulose, in range of 10-35 percent by weight, (e) glycerol monostearate, in range of 2-8 percent by weight; wherein molecules of the fluvastatin sodium are separate from one another and dispersed in a portion of the hydroxypropyl methylcellulose. In one embodiment, the hypromellose is Hypromellose 2208 commercially available from the DOW chemical company as METHOCEL™ K4M Premium. In one embodiment, the hypromellose has a methoxyl content between 19.0 and 24.0 percent by weight and a hydroxypropoxyl content between 7.0 and 12.0 percent by weight. In certain embodiments, the viscosity of 2% solution of Hypromellose in water is 2663-4970 mPa·s;

One or more polymers used in the solid dispersion composition can be any pharmaceutically acceptable polymers. Examples include water-soluble and water-insoluble polymers. Water-insoluble polymers include ethylcellulose, methacrylate copolymers (for example, Eudragits such as Eudragit E, R, S, RS and LD). Water-soluble polymers include alginate, propylene glycol alginate, carbopol, hydroxypropyl methyl cellulose (hypromellose), hydroxypropyl cellulose, polyethylene oxide, polyethylene glycol, cellulose gum, polyvinylpyrrolidone, vinylpyrrolidone/vinyl acetate copolymer and poly-propylene glycol or other similar acceptable polymers.

The pharmacologically acceptable polymer included in the total pharmaceutical composition may be in an amount of about 0.1 wt % to about 50 wt %, such as about 5 wt % to about 45 wt % of the total pharmaceutical composition. As an example, polyvinylpyrrolidone can be used to between about 5 wt % and about 50 wt %. As another example, polyethylene oxide can be used to between about 5 wt % and about 50 wt %. In another example, polyethylene glycol can be used to between about 5 wt % and about 50 wt % as a pharmacologically acceptable polymer.

Optionally, a surfactant, such as sodium lauryl sulfate and/or polyethylene glycol, can be used. For example, the surfactant can be incorporated in an amount of 0.1 wt % to about 50 wt %, such as about 5 wt % to about 45 wt % of the total pharmaceutical composition.

The solid dispersion composition can be produced by dissolving a mixture of the active ingredients and the one or more pharmaceutical acceptable polymers in a solvent and removing the solvent afterward. Alternatively, the solid dispersion composition can be produced by fusing the mixture at high temperatures and solidifying after cooling down.

In solvent-evaporation method, the solvent is selected to be able to dissolve the active ingredients and the one or more pharmaceutical acceptable polymers. Examples of the solvent may include water, polar solvent, and alcohols, suitable for a combination of dispersion components, such as fluvastatin sodium and water-soluble polymers. Examples of the solvent may include acetone and alcohols, suitable for a combination of dispersion components, such as fluvastatin sodium and water-insoluble polymers.

No particular limitation is imposed on how to remove the solvent. Examples of the various ways to remove the solvent include, but are not limited to, evaporation under reduced pressure; atomizing the solution by means of a spray dryer; and applying the solution to core particles (lactose, microcrystalline cellulose, and/or anhydrous dibasic calcium phosphate) placed in an apparatus such as a fluid bed granulator or a rotary granulator, to thereby cause the solvent to be evaporated.

The solid dispersion composition is applied in solution to additional components of the pharmaceutical compositions to form a granule, pellet or other dosage forms. Such additional components may include controlled release agents, binders, lubricant, fillers, and/or other pharmacologically acceptable carriers. Examples of these components include, but are not limited to, excipients such as lactose, microcrystalline cellulose, sucrose, mannitol, light anhydrous silicic acid, and dibasic calcium phosphate; binders such as methyl cellulose, hydroxypropyl cellulose, gelatin, polyvinylpyrrolidone, guar gum, xanthan gum, hydroxypropyl methyl cellulose, ethylcellulose, acrylates, and pullulan; lubricants such as magnesium state stearic acid, silicon dioxide, glycerol monostearate and talc; colorants such as tar pigments and red ferric oxide; and flavoring agents such as stevia, aspartame, and perfume.

No particular limitation is imposed on the dosage form for the pharmaceutical composition as described herein. However, the preferred dosage form is a tablet. In one embodiment, the tablet is optionally coated with an enteric polymer. Examples of enteric polymers include cellulose acetate phthalate, cellulose acetate succinate, methylcellulose phthalate, ethylhydroxycellulose phthalate, polyvinylacetatephthalate, polyvinylbutyrate acetate, vinyl acetate-maleic anhydride copolymer, styrene-maleic mono-ester copolymer, methyl acrylate-methacrylic acid copolymer, methacrylate-methacrylic acid-octyl acrylate copolymer, etc. The preferred enteric polymer is Poly(methacrylic acid-co-ethyl acrylate) 1:1 Dispersion.

The many features and advantages of the invention are apparent from the written description, and thus, it is intended by the appended claims to cover all such features and advantages of the invention. Further, since numerous modifications and changes will readily occur to those skilled in the art, it is not desired to limit the invention to the exact construction and operation as illustrated and described. Hence, all suitable modifications and equivalents may be resorted to as falling within the scope of the invention.

### EXAMPLES

Examples as described below should not be construed as limiting the invention thereto.

### Example 1

### Lot No. 092806B

Fluvastatin sodium, polyvinylpyrrolidone (Plasdone K-29/32, ISP), hydroxypropyl methylcellulose (Methocel™ K100 M, Dow), microcrystalline cellulose (Avicel Ph 101, FMC), and magnesium stearate (Spectrum) were blended and compressed into tablets weighted 328 milligrams (mg) at hardness of about 8 kilopond (kp) to about 11 kp. These tablets first appeared uniform in color. However, tiny spots of yellow color appeared after stored under accelerated conditions for one month. These tablets also exhibited crystalline structures as observed under a polarized microscope.

### Example 2

### Lot No. 110906

Fluvastatin sodium, sodium lauryl sulfate (Spectrum), and polyvinylpyrrolidone (Plasdone K-29/2, ISP) were co-dissolved in water to form into a dispersion solution. The prepared dispersion solution was applied in portions to a granulator having a mixture of hydroxypropyl methylcellulose (Methocel™ K100 M, Dow), microcrystalline cellulose (Avicel Ph 101, FMC), and silicon dioxide (Cab-O-Sil, Cabot) therein in order to generate granules of a solid dispersion composition. The solid dispersion composition was dried at about 55° C. until LOD (Loss on Drying) was below 3%. The granules were milled and lubricated with magnesium stearate. The final blend was then compressed into tablets. A uniform color was found on the surface of each tablet. No crystalline structure/form was observed under a polarized microscope. When the generated granule was observed under a polarized-light microscope for birefringence using a LOMO optical microscope, no birefringence was observed, indicating that the fluvastatin sodium existed in amorphous form. The tablet appeared slightly darker but yellow spots did not show up on the surface of the tablets after stored under accelerated conditions for one month.

### Example 3

### Lot No. 111306

Fluvastatin sodium, sodium lauryl sulfate (Spectrum), and polyethylene oxide (Polyox N80, Dow) were co-dissolved in water to form into a dispersion solution. The prepared dispersion solution was applied in portions to a granulator having a mixture of hydroxypropyl methylcellulose (Methocel™ K100 M, Dow), microcrystalline cellulose (Avicel Ph 101, FMC), and silicon dioxide (Cab-O-Sil, Cabot) to produce granules of a solid dispersion composition. The solid dispersion composition was dried at about 55° C. until LOD was below 3%. The granules were milled and lubricated with magnesium stearate. The final blend was then compressed into tablets. Color was uniformly distributed on tablet surface and the formula allowed a sustained-release of the fluvastatin sodium. No crystal was observed under a polarized microscope, and the fluvastatin sodium existed in amorphous form in solid dispersion composition.

### Example 4

### Lot No. 111505PEG

Fluvastatin sodium was added to a molten polyethylene glycol 3350 (Dow) at above 80° C. to form into a dispersion solution. The dispersion solution was stirred until a homogeneous state was formed and congealed into a solid dispersion form at lower temperature by cooling down to room temperature. The solid dispersion composition was milled, blended with silicon dioxide and compressed into a tablet containing 80 mg of fluvastatin sodium. The solid dispersion appeared to have a uniform color. The tablet made of this dispersion released the drug completely within an hour.

### Example 5

### Lot No. 111506

Fluvastatin sodium, sodium lauryl sulfate (Spectrum), and polyethylene glycol (Dow) were co-dissolved in water to form into a dispersion solution. The prepared dispersion solution was applied in portions to a granulator having a mixture of hydroxypropyl methylcellulose (Methocel™ K100 M, Dow), microcrystalline cellulose (Avicel Ph 101, FMC), and silicon dioxide (Cab-O-Sil, Cabot) to yield granules of a solid dispersion composition. The solid dispersion composition was dried at about 55° C. until LOD was below 3%. The granules were milled and lubricated with magnesium stearate. The final blend was then compressed into tablets. Color was uniformly distributed on a tablet surface and the formula exhibited a sustained-release profile of the fluvastatin sodium.

TABLE 1
In Vitro dissolution profiles of Examples 1-5

| | Example 1 | Example 2 | Example 3 | Example 4 | Example 5 |
|---|---|---|---|---|---|
| | (Lot No. | (Lot No. | (Lot No. | (Lot No. | (Lot No. |
| Time, hr | 092806B) | 110906) | 111306) | 111506PEG) | 111506) |
| 1 | 6 | 13.4 | 1.7 | 92.1 | 6.7 |
| 4 | 24.9 | 20.8 | 7.4 | 92.5 | 17.9 |
| 8 | 51.6 | 31.1 | 15.5 | 92.7 | 29.8 |
| 12 | 69.9 | 41.9 | 24.5 | 92.7 | 39.4 |

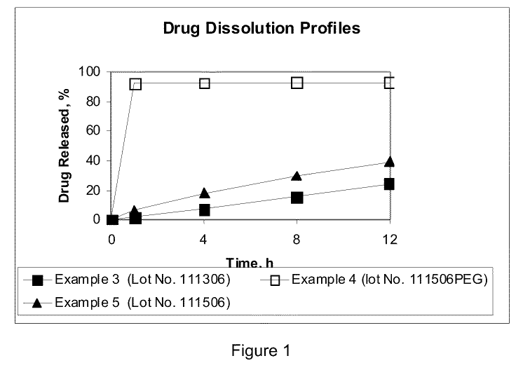

The release profiles of the pharmaceutical compositions of examples 1-5 in simulated intestinal fluid (Paddle Method 50 rpm, 37° C., n=6) is summarized in Table 1. In addition, the release profiles of the pharmaceutical compositions of examples 3, 4, and 5 are illustrated in FIG. 1. A constant release rate was observed from these examples, demonstrating a substantially zero order dissolution rate. The examples of fluvastatin-containing pharmaceutical compositions appear to include amorphous fluvastatin, and the solid dispersion composition enables fluvastatin to be constantly released over a time period, such as a period of about 12 hours.

### Example 6

Fluvastatin sodium and hydroxypropyl methylcellulose (Methocel™ K4M, Dow) was added to a water and acetone solution to form into a dispersion solution. The dispersion solution was stirred until a homogeneous state was formed. The prepared dispersion solution was applied in portions to a granulator having a mixture of cellulose gum and microcrystalline cellulose (Avicel Ph 101, Dow) to produce granules of a solid dispersion composition. The solid dispersion composition was dried at about 55° C. until LOD was below 3.4%. The granules were milled and mixed with glycerol monostearate, cellulose gum and polyethylene oxide to form a final blend. The final blend was then compressed into tablets.

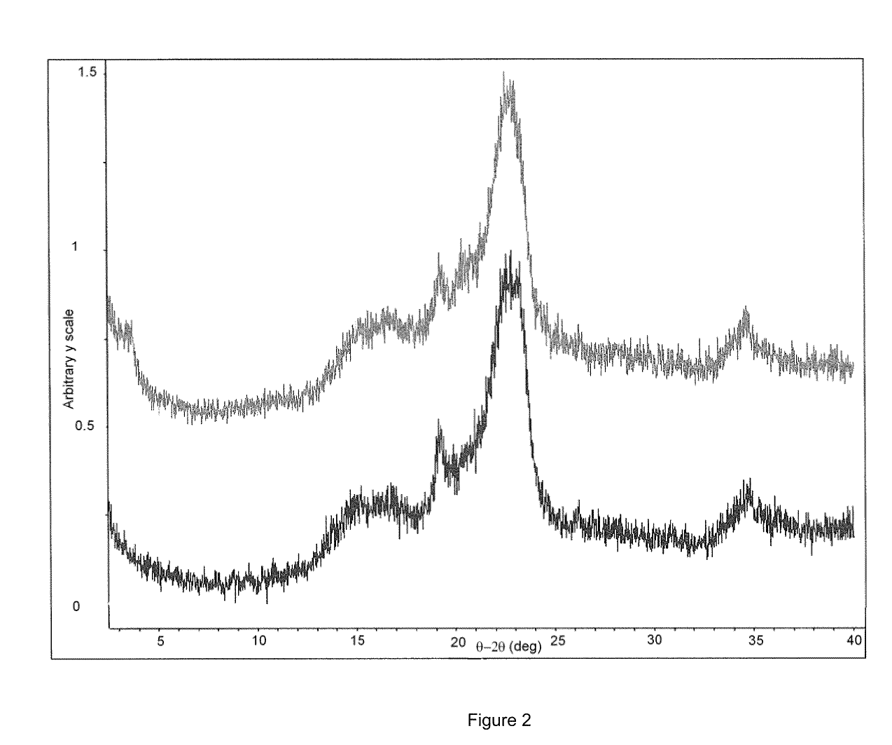

A placebo was prepared with the same process and constituents as the composition of example 6 except without the fluvastatin sodium. The placebo and the composition of example 6 were each ground to a powder and analyzed using a Shimadzu XRD-6000 X-ray powder diffractometer. FIG. 2 shows diffracted radiation results obtained from the X-ray powder diffraction analysis for the placebo (bottom profile) and the composition of example 6 (top profile). Two possible peaks observed in the profile for the composition of example 6 at about 3.5 and 20.4° 2θ were not observed in the profile for the placebo. The two peaks were from the fluvastatin sodium or its interaction with other ingredients and indicated some kind of crystalline structure/form. However, a lack of any strong peaks and the two peaks being different from any existing known form of fluvastatin sodium demonstrated that the fluvastatin sodium was amorphous.

### Example 7

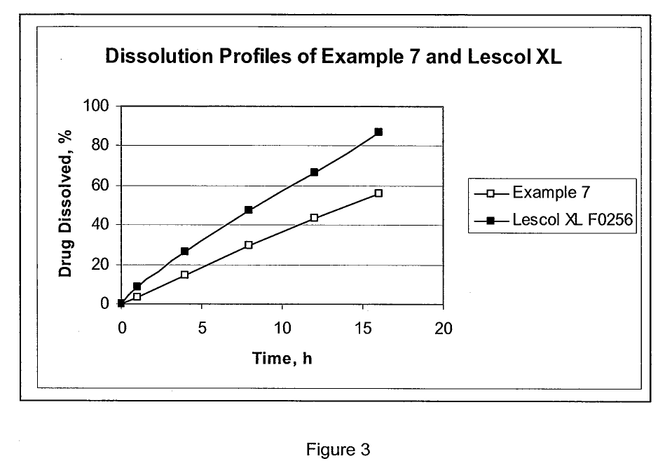

Fluvastatin sodium, 40 g, was co-dissolved with hydroxypropyl methylcellulose (Methocel™ K4M, Dow), 2.5 g, in a mixture of water and acetone to form a fluvastatin sodium solution. This fluvastatin sodium solution was sprayed onto a mixture of cellulose gum, 57.5 g, microcrystalline cellulose, 60 g and hydroxypropyl methylcellulose (Methocel™ K4M, Dow), 2.5 g, in a vertical high-shear mixer. The granules were passed through a 12 mesh-screen and dried at 50° C. till loss-on-drying (moisture content) equaled to 3.2%. The granules were milled to less than 10% of the population larger than a 20 mesh-screen opening and 10% smaller than a 100 mesh-screen opening. The milled granules, 123.5 g, were then blended with glycerol monostearate, 3.04 g, for 20 minutes. The blend was compressed into a tablet weighing 351.7 mg with a hardness of 17 Kp. The dissolution profile in simulated intestinal fluid without enzyme at pH 6.8 (basket method (75 rpm)) of this formula compared with LESCOL® XL, commercially available from NOVARTIS are illustrated in FIG. 3. In absence of food during dosing, the Gmean and AUC ratios with respect to LESCOL® XL's are 1.2 and 1.17 (n=5).

### Example 8

The composition of Example 8 is shown in Table II.

TABLE II
Composition of Example 8

| | Example 8 | |
|---|---|---|
| | Tablet, mg | % |
| Fluvastatin Sodium | 84.483 | 22.88 |
| Hydroxypropyl Methylcellulose (Methocel ™ K4M, | 8.0 | 2.17 |
| Dow) | | |
| Hydroxypropyl Methylcellulose (Methocel ™ K4M, | 40 | 10.83 |
| Dow) | | |
| Cellulose Gum | 121 | 32.77 |
| Microcrystalline Cellulose | 90 | 24.38 |
| Glycerol Monostearate | 8.45 | 2.29 |
| Enteric Coating | | |
| Poly(methacrylic acid-co-ethyl acrylate) 1:1 | 13.93 | 3.77 |
| Dispersion | | |
| Triethyl Citrate | 2.79 | 0.76 |
| Polysorbate 80 | 0.57 | 0.16 |
| Total | 369.23 | 100.0 |

*Equivalent to 80 mg of fluvastatin.

The manufacturing process of Example 8 is similar to that of Example 7; an enteric coat was applied to Example 8. In absence of food during dosing, its Gmean and AUC ratios with respect to LESCOL XL's are 0.8 and 0.89 (n=6).

While the foregoing is directed to embodiments of the invention, other and further embodiments of the invention may be devised without departing from the basic scope thereof, and the scope thereof is determined by the claims that follow.

What is claimed is:

1. A solid dispersion extended release tablet composition, comprising:
fluvastatin sodium; and
a polymer, wherein molecules of the fluvastatin sodium are separate from one another and dispersed in the polymer; and
wherein the composition displays at least one distinct peak of X ray diffraction.

2. The solid dispersion extended release tablet composition of claim 1, wherein at least a portion of the polymer comprises at least one of polyvinylpyrrolidone, polyethylene oxide, hypromellose, cellulose gum, microcrystalline cellulose and polyethylene glycol.

3. The solid dispersion extended release tablet composition of claim 1, further comprising a lubricant selected from at least one of magnesium stearate, silicon dioxide, glycerol monostearate, and talc.

4. The solid dispersion extended release tablet composition of claim 1, wherein the fluvastatin sodium is amorphous.

5. The solid dispersion extended release tablet composition of claim 1, further comprising hypromellose, cellulose gum, microcrystalline cellulose, polyethylene oxide and glycerol monostearate.

6. A solid dispersion extended release tablet composition comprising:
fluvastatin sodium; and
a polymer in a solidification of an at least partially liquid dispersion solution of the fluvastatin sodium and the polymer together, wherein at least some of both the fluvastatin sodium and the polymer are liquid in the at least partially liquid dispersion solution, wherein at least a portion of the fluvastatin sodium is dispersed in the polymer, wherein the fluvastatin sodium has an amorphous form in the solidification and wherein molecules of the fluvastatin sodium are separate from one another and dispersed in the polymer; wherein the composition enables a constant release rate of fluvastatin sodium for a period of about 12 hours.

7. The solid dispersion extended release tablet composition of claim 6, wherein the constant release rate of fluvastatin sodium is a substantially zero order dissolution rate.

8. The solid dispersion extended release tablet composition of claim 7, wherein the constant release rate was determined in simulated intestinal fluid using a Paddle Method [50 RPM, 37 degrees Celsius, 900 mL, simulated intestinal fluid without enzyme; where n=6].

9. The solid dispersion extended release tablet composition of claim 6, further comprising sodium lauryl sulfate.

10. The solid dispersion extended release tablet composition of claim 9, wherein at least a portion of the polymer comprises polyethylene glycol.

11. The solid dispersion extended release tablet composition of claim 10, wherein at least a portion of the polymer comprises polyethylene glycol.

12. A solid dispersion extended release tablet composition, comprising:
fluvastatin sodium; and
a polymer, wherein molecules of the fluvastatin sodium are separate from one another and dispersed in the polymer; and
wherein the corresponding Cₘₐₓ ratio of the composition with respect to a commercially-available fluvastatin extended-release tablet is about 0.8-1.2.

13. The solid dispersion extended release tablet composition of claim 12, further comprising glycerol monostearate.

14. An extended release tablet composition, comprising:
(a) a granule comprising a dispersion and a substrate:
(i) the dispersion comprising fluvastatin sodium and Hypromellose 2208, wherein molecules of the fluvastatin sodium are separate from one another and dispersed in the Hypromellose 2208 and wherein the viscosity of 2% solution of Hypromellose 2208 in water is 2,663-4,970 mPa·s; and
(ii) the substrate comprising cellulose gum and microcrystalline cellulose, wherein the dispersion and substrate are combined to form granules; and
(b) extra-granular materials comprising glycerol monostearate, cellulose gum and polyethylene, wherein the extended release tablet composition displays two distinct peaks at about 3.5 and 20.4.degrees 26 of X-ray diffraction and wherein the composition enables fluvastatin to be constantly released over a time period of about 12 hours.

15. An extended release tablet composition, comprising:
(a) a granule comprising a dispersion and a substrate:
(i) the dispersion comprising fluvastatin sodium and Hypromellose 2208, wherein molecules of the fluvastatin sodium are separate from one another and dispersed in the Hypromellose 2208; and
(ii) the substrate comprising microcrystalline cellulose, cellulose gum and Hypromellose 2208; and
(b) extra-granular materials comprising glycerol monostearate, wherein the viscosity of 2% solution of Hypromellose 2208 in water is 2,663-4,970 mPa·s; and wherein the corresponding Cₘₐₓ ratio of the composition with respect to a commercially-available fluvastatin extended-release tablet is about 0.8-1.2.

16. The extended release tablet composition of claim 15, wherein the extended release tablet composition is coated with an enteric polymer.

17. The extended release tablet composition of claim 16, wherein the enteric polymer is poly(methacrylic acid-co-ethyl acrylate) 1:1 dispersion.

18. An extended release tablet composition, comprising:
(a) a granule comprising a dispersion and a substrate:
(i) the dispersion comprising fluvastatin sodium and Hypromellose 2208, wherein molecules of the fluvastatin sodium are separate from one another and dispersed in the Hypromellose 2208;
(ii) the substrate comprising microcrystalline cellulose, cellulose gum and Hypromellose 2208; and
(b) extra-granular materials comprising glycerol monostearate, wherein the viscosity of 2% solution of Hypromellose 2208 in water is 2,663-4,970 mPa·s and wherein the composition enables a constant release rate of fluvastatin sodium for a period of about 12 hours.

19. The solid dispersion extended release tablet composition of claim 18, wherein the constant release rate of fluvastatin sodium is a substantially zero order dissolution rate.

20. The solid dispersion extended release tablet composition of claim 19, wherein the constant release rate was determined in simulated intestinal fluid using a Paddle Method [50 RPM, 37 degrees Celsius, 900 mL, simulated intestinal fluid without enzyme; where n=6].

21. An extended-release tablet composition comprising:
(a) about 84.5 mg of fluvastatin sodium;
(b) hypromellose 2208 in range of 2-35 percent by weight;
(c) cellulose gum in range of 20-50 percent by weight;
(d) microcrystalline cellulose, in range of 10-35 percent by weight, and
(e) glycerol monostearate, in range of 2-8 percent by weight; wherein molecules of the fluvastatin sodium are separate from one another and dispersed in a portion of the Hypromellose 2208; wherein the composition is optionally enteric-coated and wherein the constant release rate of fluvastatin sodium is a substantially zero order dissolution rate and wherein the corresponding Cₘₐₓ ratio of the composition with respect to a commercially-available fluvastatin extended-release tablet is about 0.8-1.2.
